(19) United States
(12) Patent Application Publication
(10) Pub. No.: US 2025/0115638 A1
(43) Pub. Date: Apr. 10, 2025

(54) PEPTIDE ANTIGENS FOR FORMYLMETHIONINE ANTIBODY PRODUCTION

(71) Applicants: Korea University Research and Business Foundation, Seoul (KR); POSTECH Research and Business Development Foundation, Pohang-si (KR); UNIVERSITY INDUSTRY FOUNDATION, YONSEI UNIVERSITY WONJU CAMPUS, Wonju-si (KR)

(72) Inventors: Cheol-Sang HWANG, Seoul (KR); Dasom KIM, Seoul (KR); Kyu-Sang PARK, Wonju-si (KR)

(73) Assignees: Korea University Research and Business Foundation, Seoul (KR); POSTECH Research and Business Development Foundation, Pohang-si (KR); UNIVERSITY INDUSTRY FOUNDATION, YONSEI UNIVERSITY WONJU CAMPUS, Wonju-si (KR)

(21) Appl. No.: 18/910,211

(22) Filed: Oct. 9, 2024

(30) Foreign Application Priority Data
Oct. 10, 2023 (KR) 10-2023-0134436

(51) Int. Cl. C07K 5/08; C07K 16/06; C07K 16/44

(52) U.S. Cl. CPC C07K 5/08; C07K 16/065; C07K 16/44

(57) ABSTRACT

Provided is a novel mixed peptide antigen for production of a formylmethionine antibody. According to the present disclosure, the formylmethionine-specific antibody produced using the antigen peptide can detect formylmethionine by universally binding to peptides or proteins containing formylmethionine, and has higher sensitivity than antibodies using conventional single antigen peptides.

### CROSS-REFERENCE TO RELATED APPLICATIONS

This application is based on and claims priority from Korean Patent Application No. 10-2023-0134436, filed on Oct. 10, 2023, with the Korean Intellectual Property Office, the disclosure of which is incorporated herein in its entirety by reference.

### TECHNICAL FIELD

The present disclosure relates to a novel mixed peptide antigen for production of a formylmethionine antibody.

### BACKGROUND

Methionine is a common starting amino acid residue for extending peptide chains (e.g., protein synthesis) in most living systems. Two of the most notable modifications of the initiating amino acid, methionine, include the addition of an amino (N-) formyl group to methionine molecules prior to the initiation of messenger RNA (mRNA) translation and the subsequent removal of the N-formyl group from methionine at an N-terminus of an immature peptide. N-terminus formylation is a type of protein modification, and is known to be mainly observed in bacteria, mitochondria, and chloroplasts, and observed even in the cytoplasm of eukaryotic cells under specific stress situations. An enzyme that plays a major role in the formylation of intracellular proteins is formylmethionyl-transferase, and this enzyme attaches a formyl group to methionyl-tRNA to produce formylmethionyl-tRNA. Thereafter, when the formylmethionyl-tRNA is used to initiate protein synthesis, proteins containing formylmethionine at the N-terminus are synthesized. The N-formyl group is removed by peptide deformylase (PDF). PDF cleaves the formyl group from most immature formylmethionine-peptides in a substrate-specific reaction. However, there are exceptions to the general action of PDF. For example, some E. coli proteins exist in a partially formylated form (Hauschild-Rogat, P., 1968; Marasco, W. A., et al., 1984; and Milligan, D. L. and Koshland, Jr., D. E., 1990).

Meanwhile, methods for producing antibodies to specifically detect the N-terminus formylation of proteins have been proposed a long time ago. For example, in order to produce an antibody that recognizes a peptide called formyl-MLF, a method for producing an antibody by attaching a formyl-MLF-like peptide to a carrier protein to be used as an antigen has been used. However, when the antibody is produced using a single peptide antigen as described above, the antibody has a disadvantage of strongly recognizing only peptides/proteins that have a similar structure or sequence to the peptide antigen, and weakly or not recognizing peptides/proteins that have a different structure or sequence from the peptide antigen. For example, recently, in the case of a polyclonal antibody produced using fMGSGC as an antigen, the polyclonal antibody strongly recognizes peptides starting with fMG, but relatively weakly recognizes other peptides starting with fM, which also acts as a cause of lowering the sensitivity of the antibody itself. Therefore, when using existing methods, antibodies recognizing each formyl peptide need to be prepared separately, and when the diversity of protein sequences is considered, inefficiency becomes noticeable.

Accordingly, there is a need to develop an antibody capable of overcoming the limitations of the prior art and universally recognizing peptides/proteins.

### SUMMARY

The present disclosure has been made in an effort to provide an antigen peptide for producing a formylmethionine (fMet, fM)-specific antibody.

The present disclosure has also been made in an effort to provide a composition for producing a formylmethionine-specific antibody.

The present disclosure has also been made in an effort to provide a kit for producing a formylmethionine-specific antibody.

The present disclosure has also been made in an effort to provide a method for producing a formylmethionine-specific antibody.

The present disclosure has also been made in an effort to provide a formylmethionine-specific antibody.

The present disclosure has also been made in an effort to provide a composition for detecting formylmethionine.

The present disclosure has also been made in an effort to provide a kit for detecting formylmethionine.

The present disclosure has also been made in an effort to provide a method for detecting a peptide or protein containing formylmethionine.

An exemplary embodiment of the present disclosure provides an antigen peptide for producing a formylmethionine-specific antibody.

Another exemplary embodiment of the present disclosure provides a composition for producing a formylmethionine-specific antibody including the antigen peptide.

Yet another exemplary embodiment of the present disclosure provides a kit for producing a formylmethionine-specific antibody including the antigen peptide.

Still another exemplary embodiment of the present disclosure provides a method for producing a formylmethionine-specific antibody.

Still another exemplary embodiment of the present disclosure provides a formylmethionine-specific antibody.

Still another exemplary embodiment of the present disclosure provides a composition for detecting formylmethionine including the antibody.

Still another exemplary embodiment of the present disclosure provides a kit for detecting formylmethionine including the antibody.

Still another exemplary embodiment of the present disclosure provides a method for detecting a peptide or protein containing formylmethionine.

According to the exemplary embodiments of the present disclosure, the formylmethionine-specific antibody produced using the antigen peptide can detect formylmethionine by universally binding to a peptide or protein containing formylmethionine, and has higher sensitivity than antibodies using conventional single antigen peptides.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

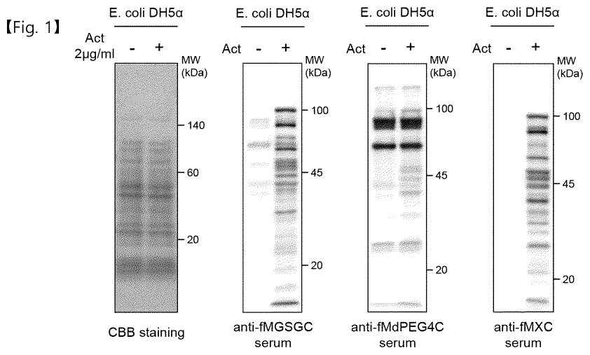

FIG. 1 is a diagram confirming antigen-binding ability to formylmethionine of a formylmethionine antibody of the present disclosure in E. coli:
Act: Actinonin;
CBB staining: Coomassie Brilliant Blue staining image;
Anti-fMGSGC serum: Serum containing antibody produced using conventional fMGSGC antigen;
Anti-fMdPEG4C serum: Serum containing antibody produced using fMdPEG4C antigen; and
Anti-fMXC serum: Serum containing antibody produced using mixed antigen fMXC.

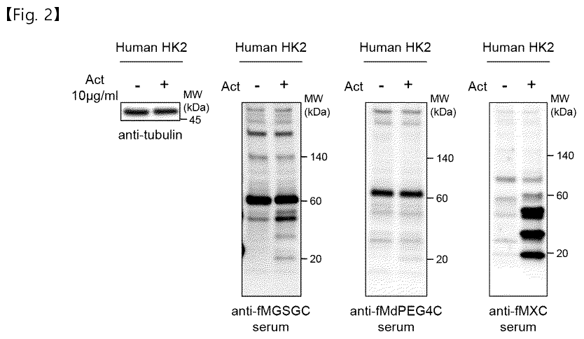

FIG. 2 is a diagram confirming antigen-binding ability to formylmethionine of a formylmethionine antibody of the present disclosure in mammalian cells:
Act: Actinonin;
Anti-fMGSGC serum: Serum containing antibody produced using conventional fMGSGC antigen;
Anti-fMdPEG4C serum: Serum containing antibody produced using fMdPEG4C antigen; and
Anti-fMXC serum: Serum containing antibody produced using mixed antigen fMXC.

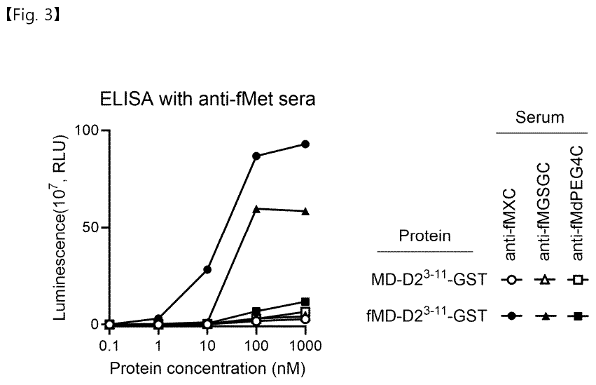

FIG. 3 is a diagram confirming that the antibody produced using the mixed antigen fMXC exhibits significantly higher antibody sensitivity compared to the antibodies produced using the conventional fMGSGC antigen and the fMdPEG4C antigen.
Anti-fMGSGC serum: Serum containing antibody produced using conventional fMGSGC antigen;
Anti-fMdPEG4C serum: Serum containing antibody produced using fMdPEG4C antigen; and
Anti-fMXC serum: Serum containing antibody produced using mixed antigen fMXC.

### DETAILED DESCRIPTION

Hereinafter, exemplary embodiments of the present disclosure will be described in detail. However, the following exemplary embodiments are presented as examples of the present disclosure, and the present disclosure is not limited thereby, and various modifications and applications of the present disclosure can be made within the description of claims to be described below and equivalents interpreted therefrom.

Unless defined otherwise, all technical and scientific terms used herein have the same meaning as commonly understood by those skilled in the art to which the present disclosure pertains. Although any methods and materials similar or equivalent to those described herein can be used in the practice for testing the present disclosure, the preferred materials and methods are described herein.

Throughout the present specification, general one-letter or three-letter codes for naturally existing amino acids are used, and generally allowed three-letter codes for other amino acids, such as a-aminoisobutyric acid (Aib) and N-methylglycine (Sar) are also used. The amino acids mentioned herein as abbreviations are also described as follows according to the IUPAC-IUB nomenclature.

Alanine: Ala, A; Arginine: Arg, R; Asparagine: Asn, N; Aspartic acid: Asp, D; Cysteine: Cys, C; Glutamic acid: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan: Trp, W; Tyrosine: Tyr, Y; and Valine: Val, V.

In an aspect, the present disclosure relates to an antigen peptide for producing a formylmethionine (fMet, fM)-specific antibody, including an amino acid sequence of Formula 1 below:

fM-X-C (Formula 1, SEQ ID NO: 1)
wherein,
fM is formylmethionine; and
X is G (Gly), A (Ala), V (Val), L (Leu), I (Ile), T (Thr), S (Ser), C (Cys), M (Met), D (Asp), N (Asn), E (Glu), Q (Gln), K (Lys), R (Arg), H (His), F (Phe), Y (Tyr), W (Trp) or P (Pro).

In an exemplary embodiment, X may be at least one selected from the group consisting of G (Gly), A (Ala), V (Val), L (Leu), I (Ile), T (Thr), S (Ser), C (Cys), M (Met), D (Asp), N (Asn), E (Glu), Q (Gln), K (Lys), R (Arg), H (His), F (Phe), Y (Tyr), W (Trp), and P (Pro).

In an exemplary embodiment, the antigen peptide of the present disclosure may include a peptide including an amino acid sequence of fMGC, a peptide including an amino acid sequence of fMAC, a peptide including an amino acid sequence of fMVC, a peptide including an amino acid sequence of fMLC, a peptide including an amino acid sequence of fMIC, a peptide including an amino acid sequence of fMTC, a peptide including an amino acid sequence of fMSC, a peptide including an amino acid sequence of fMCC, a peptide including an amino acid sequence of fMMC, a peptide including an amino acid sequence of fMDC, a peptide including an amino acid sequence of fMNC, a peptide including an amino acid sequence of fMEC, a peptide including an amino acid sequence of fMQC, a peptide including an amino acid sequence of fMKC, a peptide including an amino acid sequence of fMRC, a peptide including an amino acid sequence of fMHC, a peptide including an amino acid sequence of fMFC, a peptide including an amino acid sequence of fMYC, a peptide including an amino acid sequence of fMWC, or a peptide including an amino acid sequence of fMPC.

In an exemplary embodiment, the antigen peptide of the present disclosure may be a mixed peptide including fMGC (SEQ ID NO: 2), fMAC (SEQ ID NO: 3), fMVC (SEQ ID NO: 4), fMLC (SEQ ID NO: 5), fMIC (SEQ ID NO: 6), fMTC (SEQ ID NO: 7), fMSC (SEQ ID NO: 8), fMCC (SEQ ID NO: 9), fMMC (SEQ ID NO: 10), fMDC (SEQ ID NO: 11), fMNC (SEQ ID NO: 12), fMEC (SEQ ID NO: 13), fMQC (SEQ ID NO: 14), fMKC (SEQ ID NO: 15), fMRC (SEQ ID NO: 16), fMHC (SEQ ID NO: 17), fMFC (SEQ ID NO: 18), fMYC (SEQ ID NO: 19), fMWC (SEQ ID NO: 20) and fMPC (SEQ ID NO: 21).

In an exemplary embodiment, the antigen peptide may further include a carrier protein, and the carrier protein may be Keyhole-Limpet Hemocyanin (KLH), bovine serum albumin (BSA), or ovalbumin (OVA).

In an exemplary embodiment, the carrier protein may be linked to the antigen peptide via a linker, and the linker may be a chemical linker and may be sulfosuccinimidyl 4-(N-maleimidomethyl)cyclohexane-1-carboxylate (sulfo-SMCC).

In an exemplary embodiment, the carrier protein may be bound/attached to cysteine of the antigen peptide.

In an exemplary embodiment, the carrier protein may be bound/attached to a C-terminus of the antigen peptide and may be bound/attached to a carboxyl group of the C-terminus.

In an exemplary embodiment, a cysteine (Cys) group of the antigen peptide and a lysine (Lys) group of the carrier protein, Keyhole-Limpet Hemocyanin may be bound/linked to each other using Sulfo-SMCC.

In the present disclosure, the formylation refers to the action of introducing a formyl group (CHO—) into a compound. In the present disclosure, the “formylation” refers to formylation of an α-amino group of an amino acid.

As used herein, the “peptide” is a polymer of amino acids, and usually, a form in which a small number of amino acids are linked is called a peptide, and a form in which many amino acids are linked is called a protein. The linkage between amino acids in a peptide or protein structure consists of amide bonds or peptide bonds. The peptide bond refers to a bond in which water (H₂O) is removed between a carboxyl group (—COOH) and an amino group (—NH₂) to form —CO—NH—. The peptide of the present disclosure may be prepared according to chemical synthesis methods known in the art, especially solid-phase synthesis techniques (Merrifield, J. Amer. Chem. Soc. 85:2149-54(1963); Stewart, et al., Solid Phase Peptide Synthesis, 2nd. ed., Pierce Chem. Co.: Rockford, 111 (1984)), and also produced by genetic manipulation techniques.

In the present disclosure, in the modification of the peptide sequence, some amino acids may be modified through any one of substitution, addition, deletion, and modification, or a combination of these methods. These modifications include modifications using L-type or D-type amino acids, and/or non-natural amino acids; and/or modifications by modifying a native sequence, such as modification of side-chain functional groups, intramolecular covalent bonding, such as inter-side chain ring formation, methylation, acylation, ubiquitination, phosphorylation, aminohexation, biotinylation, etc. As the substituted or added amino acids, not only 20 amino acids conventionally observed in human proteins, but also atypical or non-naturally occurring amino acids may be used. Commercial sources of the atypical amino acids include Sigma-Aldrich, ChemPep, and Genzyme pharmaceuticals. The sequences for the peptide including these amino acids and the typical peptide can be synthesized and purchased through commercialized peptide synthesis companies, such as American Peptide Company or Bachem in USA, or Anygen in Korea.

In addition, the range of the antigen peptide of the present disclosure includes functional equivalents of the peptide containing the amino acid sequence of SEQ ID NO: 1, more preferably functional equivalents of the peptide containing the amino acid sequence of SEQ ID NO: 1, and salts thereof.

The functional equivalent refers to a peptide which has sequence homology (that is, identity) of at least 75% or more, preferably 90%, and more preferably 95% or more with the peptide of SEQ ID NO: 1, for example, sequence homology of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%, as a result of addition, substitution or deletion of amino acids, and exhibits substantially the same physiological activity as the peptide of SEQ ID NO: 1. As used herein, the sequence homology and homogeneity are defined as a percentage of amino acid residues of a candidate sequence to the amino acid sequence of SEQ ID NO: 1 after aligning the amino acid sequence of SEQ ID NO: 1 and a candidate sequence and introducing gaps. If necessary, conservative substitution is not considered as a part of sequence homogeneity in order to obtain the maximum percentage sequence homogeneity. The N-terminus, C-terminus or internal extension, deletion or insertion of the amino acid sequence of SEQ ID NO: 1 is not construed as a sequence affecting sequence homology or homogeneity.

In addition, the sequence homogeneity may be determined by general standard methods used for comparing similar portions of amino acid sequences of two polypeptides. A computer program such as BLAST or FASTA aligns the two polypeptides so as to optimally match respective amino acids (according to a full-length sequence of one or two sequences, or a predicted portion of one or two sequences). The program provides a default opening penalty and a default gap penalty and provides a scoring matrix such as PAM250 (standard scoring matrix) which may be used in association with the computer program. For example, the percentage homogeneity may be calculated as follows. The total number of identical matches is multiplied by 100 and then divided into a sum of the length of a longer sequence in a corresponding matched span and the number of gaps introduced into the longer sequence to align the two sequences.

The scope of the functional equivalents of the present disclosure includes derivatives and analogues (mimetics/peptidomimetics). The “derivative” refers to a general term for similar peptides that have some modified chemical structures while maintaining the basic skeleton of the peptide of SEQ ID NO: 1, and preferably, modified chemical structures in which one or more amino acids may be substituted with another amino acid, one or more amino acids may be added, one or more amino acids may be deleted, or a compound (e.g., polyethylene glycol, etc.) that increases the half-life of the peptide may be fused. As used herein, the derivative may maintain, increase, or decrease the stability, storability, volatility, solubility, or the like of the peptide according to the present disclosure.

As used herein, the “antibody” refers to a functional ingredient of serum and is often referred to as a collection of molecules (antibodies or immunoglobulin) or a single molecule (antibody molecule or immunoglobulin molecule). The antibody molecule may bind to or react with a specific antigenic determinant (antigen or antigenic epitope) to sequentially induce an immunological effector mechanism. An individual antibody molecule is generally considered to be monospecific, and a composition of antibody molecules may be monoclonal (i.e., composed of identical antibody molecules) or polyclonal (i.e., composed of different antibody molecules that react with the same or different epitopes on the same antigen or on separate antigens). Each antibody molecule has a unique structure capable of binding specifically to a corresponding antigen thereto, and all natural antibody molecules have the same overall basic structure of two identical light chains and two identical heavy chains. The antibody is also collectively known as immunoglobulin. As used herein, the term antibody or antibodies is used in the broadest sense and includes not only intact antibodies, chimeric antibodies, humanized antibodies, complete human and single chain antibodies, but binding fragments of the antibody, such as Fab, Fv fragments or scFv fragments, and multimeric forms, such as dimeric IgA molecules or pentavalent IgM.

As used herein, the “epitope” is generally used to describe a portion of a larger molecule or a part of a larger molecule (e.g., antigen or antigenic site) that has antigenic or immunogenic activity in an animal, preferably a mammal, and most preferably a human. The epitope having immunogenic activity is a portion of a larger molecule that causes an antibody response in an animal. The epitope having the antigenic activity is a portion of a larger molecule to which an antibody immunospecifically binds, as determined by any method well known in the art, for example, by immunoassay described herein. The antigenic epitope is not necessarily immunogenic. The antigen is a substance to which an antibody or antibody fragment immunospecifically binds, such as toxin, virus, bacteria, protein, or DNA. The antigen or antigenic site often has more than one epitope, unless very small, and may often stimulate an immune response. Antibodies that bind to different epitopes on the same antigen may have various effects on the activity of antigens to which these antibodies bind, depending on a location of the epitope. An antibody that binds to an epitope at an active site of the antigen completely blocks a function of the antigen, whereas another antibody that binds to a different epitope may have little or no effect on the activity of the antigen.

In the present disclosure, the “polyclonal antibody” refers to a composition of different (various) antibody molecules capable of binding to or reacting with several different specific antigenic determinants/epitopes on the same or different antigens, and each antibody in the composition may react with a specific epitope. In general, the variability of the polyclonal antibody is present in so-called variable regions of the polyclonal antibody, particularly CDR1, CDR2 and CDR3 regions. In the present disclosure, the polyclonal antibody may be produced in one pot or may be a mixture of different polyclonal antibodies. A mixture of monoclonal antibodies is produced in individual batches and is not necessarily derived from the same organism or cell line, which would result in differences in post-translational modifications, and is not considered as a polyclonal antibody in itself. However, if the mixture of monoclonal antibodies provides the same antigen/epitope coverage as the polyclonal antibody of the present disclosure, it would be considered equivalent to the polyclonal antibody.

In an aspect, the present disclosure relates to an antigen peptide mixture consisting of a peptide including an amino acid sequence of fMGC, a peptide including an amino acid sequence of fMAC, a peptide including an amino acid sequence of fMVC, a peptide including an amino acid sequence of fMLC, a peptide including an amino acid sequence of fMIC, a peptide including an amino acid sequence of fMTC, a peptide including an amino acid sequence of fMSC, a peptide including an amino acid sequence of fMCC, a peptide including an amino acid sequence of fMMC, a peptide including an amino acid sequence of fMDC, a peptide including an amino acid sequence of fMNC, a peptide including an amino acid sequence of fMEC, a peptide including an amino acid sequence of fMQC, a peptide including an amino acid sequence of fMKC, a peptide including an amino acid sequence of fMRC, a peptide including an amino acid sequence of fMHC, a peptide including an amino acid sequence of fMFC, a peptide including an amino acid sequence of fMYC, a peptide including an amino acid sequence of fMWC, and a peptide including an amino acid sequence of fMPC.

In an exemplary embodiment, each peptide in the antigen peptide mixture may be an isolated (e.g., synthetic and/or purified) peptide.

As used herein, the peptide of the “peptide mixture” means a peptide that consists of a peptide functioning as an antigen and has a length of 3 amino acids. Accordingly, the “peptide mixture” means a mixture of the peptides.

In one aspect, the present disclosure relates to a composition for producing a formylmethionine-specific antibody including the antigen peptide of the present disclosure or the antigen peptide mixture.

In an exemplary embodiment, the composition may include an antigen peptide mixture including a peptide including an amino acid sequence of fMGC, a peptide including an amino acid sequence of fMAC, a peptide including an amino acid sequence of fMVC, a peptide including an amino acid sequence of fMLC, a peptide including an amino acid sequence of fMIC, a peptide including an amino acid sequence of fMTC, a peptide including an amino acid sequence of fMSC, a peptide including an amino acid sequence of fMCC, a peptide including an amino acid sequence of fMMC, a peptide including an amino acid sequence of fMDC, a peptide including an amino acid sequence of fMNC, a peptide including an amino acid sequence of fMEC, a peptide including an amino acid sequence of fMQC, a peptide including an amino acid sequence of fMKC, a peptide including an amino acid sequence of fMRC, a peptide including an amino acid sequence of fMHC, a peptide including an amino acid sequence of fMFC, a peptide including an amino acid sequence of fMYC, a peptide including an amino acid sequence of fMWC, and a peptide including an amino acid sequence of fMPC.

In an exemplary embodiment, the composition may be a reagent composition for producing a formylmethionine-specific antibody.

In an exemplary embodiment, the formylmethionine-specific antibody may detect the first formylmethionine regardless of a second amino acid type of the peptide or protein.

In an aspect, the present disclosure relates to a kit for producing a formylmethionine-specific antibody including the antigen peptide of the present disclosure, the antigen peptide mixture, or the composition.

The composition and kit for producing the antibody according to the present disclosure may further include known ingredients for maintaining and preserving the antigen peptide.

In an embodiment of the present disclosure, the composition and kit for producing the antibody may further include known agents for inducing and promoting an immune response in a host.

In an aspect, the present disclosure relates to a method for producing a formylmethionine-specific antibody, including inducing an immune response by inoculating the antigen peptide of the present disclosure or the antigen peptide mixture multiple times to a host other than a human; and obtaining serum from the blood of the host.

In an exemplary embodiment, the host may be a mammal other than a human, and the mammal other than the human may be mouse, rabbit, rat, guinea pig, horse, dog, sheep, goat, cat, chicken, duck, monkey or primate, and more preferably rabbit, but is not limited thereto.

In an exemplary embodiment, the serum may include a formylmethionine antibody.

In an exemplary embodiment, the method may further include purifying the formylmethionine antibody from the serum.

In an aspect, the present disclosure relates to a formylmethionine-specific antibody produced by the method of the present disclosure.

In an exemplary embodiment, the antibody may be a polyclonal antibody.

In an exemplary embodiment, the antibody may specifically bind to a peptide or protein containing formylmethionine, the formylmethionine may be the first amino acid of the peptide or protein, and the formylmethionine-specific antibody of the present disclosure may detect the first formylmethionine regardless of a second amino acid type of the peptide or protein.

In an aspect, the present disclosure relates to a composition for detecting formylmethionine including the antibody of the present disclosure.

In an exemplary embodiment, the composition may be a reagent composition.

In an aspect, the present disclosure relates to a kit for detecting formylmethionine including an antibody of the present disclosure.

In an aspect, the present disclosure relates to a method for detecting a peptide or protein containing formylmethionine including treating a sample with an antibody of the present disclosure to induce an antigen-antibody reaction.

In an exemplary embodiment, the peptide or protein may be a peptide or protein in which the first amino acid is formylmethionine.

Hereinafter, the present disclosure will be described in more detail with reference to the following Examples. However, the following Examples are only intended to embody the contents of the present disclosure, and the present disclosure is not limited thereto.

### Example 1. Production of fMXC Mixed Peptide Antigen

To produce a formylmethionine (fMet)-specific antibody, fMXC peptides were synthesized to produce a mixed antigen. Specifically, the first amino acid fM of the peptide antigen was formylmethionine, and the second amino acid X was one of glycine (Gly, G), alanine (Ala, A), valine (Val, V), leucine (Leu, L), isoleucine (Ile, I), threonine (Thr, T), serine (Ser, S), cysteine (Cys, C), methionine (Met, M), aspartic acid (Asp, D), asparagine (Asn, N), glutamic acid (Glu, E), glutamine (Gln, Q), lysine (Lys, K), arginine (Arg, R), histidine (His, H), phenylalanine (Phe, F), tyrosine (Tyr, Y), tryptophan (Trp, W), and proline (Pro, P), and the third amino acid, a crosslinkable residue, was cysteine (C). A mixed antigen fMXC was produced by mixing and synthesizing 20 kinds of antigen peptides (fMGC, fMAC, fMVC, fMLC, fMIC, fMTC, fMSC, fMCC, fMMC, fMDC, fMNC, fMEC, fMQC, fMKC, fMRC, fMHC, fMFC, fMYC, fMWC, and fMPC) having the three amino acids. In addition, a Keyhole Limpet Hemocyanin (KLH) carrier protein was attached to the synthesized peptide using a chemical linker, sulfosuccinimidyl 4-(N-maleimidomethyl)cyclohexane-1-carboxylate (sulfo-SMCC), to ultimately produce fMXC-(sulfo-SMCC)-KLH.

### Example 2. Production of Formylmethionine-Specific Antibody

The mixed antigen fMXC-(sulfo-SMCC)-KLH produced in Example 1 and its control antigens fMGSGC-(sulfo-SMCC)-KLH and fMdPEG4-(sulfo-SMCC)-KLH were each mixed with a complete Freund's adjuvant (CFA) and injected subcutaneously into rabbits (New Zealand white). Immune boosting was performed using the same method at weeks 4, 6, and 8 from an initial immunization point, and final serums (anti-fMGSGC serum, anti-fMdPEG4C serum, and anti-fMXC serum) containing antibodies were obtained by collecting blood from the heart at week 9.

### Example 3. Confirmation of Antigen-Binding Ability of Formylmethionine-Specific Antibody

### 3-1. Antigen-Binding Ability of Antibodies in E. Coli

In order to confirm the antigen-binding ability of the three types of formylmethionine-specific antibodies (anti-fMGSGC serum, anti-fMdPEG4C serum, and anti-fMXC serum) produced in Example 2 in E. coli, Coomassie blue staining and Western blot analysis were performed. Specifically, E. coli DH5α was inoculated into an LB medium (LB broth 25 g/L) and cultured overnight in an incubator at 37° C. The following day, the culture solution was diluted 100-fold in the LB medium and cultured again, and then when OD600 reached 0.6, ethanol or actinonin (Act), a peptide deformylase inhibitor, was treated for 30 minutes. At this time, actinonin was dissolved and prepared in ethanol at a concentration of 20 mg/ml and treated to E. coli to a final concentration of 2 μg/ml. Thereafter, the culture solution was centrifuged at 12,000 g for 2 minutes at 4° C. to obtain an E. coli pellet, and then the pellet was dissolved in a 2% SDS solution containing a protease inhibitor cocktail and 20 μg/ml actinonin. The pellet was sonicated for several seconds and then centrifuged at 12,000 g for 10 minutes at 4° C. to obtain a supernatant, and its protein concentration was measured using a BCA protein assay. The supernatant diluted to a concentration of 2 μg/μl was mixed with a 4× SDS sample buffer in a ratio of 3:1 (v/v), heated at 70° C. for 10 minutes, and a membrane obtained from the supernatant by electrophoresis was blocked for 1 hour with 5% skim milk (in PBS-T [137 mM NaCl, 2.7 mM KCl, 10 mM Na₂HPO₄, 1.8 mM KH₂PO₄, and 0.1% Tween 20 {w/v}]). The formylmethionine-specific antibody (anti-fMXC serum) (serum that had not undergone a purification process) produced in Example 2 and two types of control antibodies (anti-fMGSGC serum and anti-fMdPEG4C serum) diluted 1:1000 in 5% skim milk were reacted with the membrane overnight at 4° C., respectively. Thereafter, the membrane was washed three times for 10 minutes each with PBS-T, reacted with a secondary antibody (Goat anti-rabbit IgG HRP conjugate, diluted 1:5000 in 5% skim milk) for 1 hour, and then washed three times for 10 minutes each with PBS-T. Formylmethionine was detected on the washed membrane in a chemiluminescence mode using a GE Amersham Imager AI680 instrument and an HRP substrate.

As a result, it was shown that the formylmethionine antibody produced using the mixed antigen fMXC specifically bound to a protein containing formylmethionine produced in E. coli (FIG. 1). In addition, even when the production of proteins containing formylmethionine was increased in E. coli by actinonin treatment, both antibodies produced using a conventional fMGSGC antigen and antibodies produced using the mixed antigen fMXC of the present disclosure exhibited high antigen-binding ability (FIG. 1). In contrast, antibodies produced using the fMdPEG4C antigen exhibited relatively low antigen-binding ability compared to the antibodies produced using the mixed antigen fMXC (FIG. 1).

### 3-2. Antigen-Binding Ability of Antibodies in Mammalian Cells

In order to confirm the antigen-binding ability of the three types of formylmethionine-specific antibodies (anti-fMGSGC serum, anti-fMdPEG4C serum, and anti-fMXC serum) produced in Example 2 in mammalian cells, Western blot analysis was performed. Specifically, a HK2 cell line, which was human kidney-derived cells, was cultured using a DMEM/F-12+10% FBS medium, and then treated with ethanol or actinonin, a peptide deformylase inhibitor, for 10 hours, when the cell density reached 90%. At this time, actinonin was dissolved and prepared in ethanol at a concentration of 20 mg/ml and treated to HK2 cells to a final concentration of 10 μg/ml. Thereafter, cells were harvested using a trypsin enzyme and lysed in an RIPA buffer containing a protease inhibitor cocktail and 20 μg/ml actinonin. The cells were sonication for several seconds, and then centrifuged at 12,000 g for 10 minutes at 4° C. to obtain the supernatant, and the supernatant was diluted to a protein concentration of 2 μg/μl, mixed with a 4× SDS sample buffer in 3:1 (v/v) and heated at 70° C. for 10 minutes. The membrane obtained from the supernatant by electrophoresis was blocked with 5% skim milk for 1 hour, and the formylmethionine-specific antibody (anti-fMXC serum) (serum that had not undergone a purification process) produced in Example 2 and two types of control antibodies (anti-fMGSGC serum and anti-fMdPEG4C serum) were diluted 1:1000 in 5% skim milk and reacted with the membrane overnight at 4° C., respectively. Thereafter, the membrane was washed three times for 10 minutes each with PBS-T, reacted with a secondary antibody (Goat anti-rabbit IgG HRP conjugate, diluted 1:5000 in 5% skim milk) for 1 hour, and then washed three times for 10 minutes each with PBS-T. Formylmethionine was detected on the washed membrane in a chemiluminescence mode using a GE Amersham Imager AI680 instrument and an HRP substrate.

As a result, it was shown that the formylmethionine antibody produced using the mixed antigen fMXC specifically bound to a protein containing formylmethionine produced in the cells (FIG. 2). In addition, in the case of human-derived cells, since the synthesis of proteins containing formylmethionine occurred mainly only in the mitochondria, antibodies produced using the conventional fMGSGC antigen and antibodies produced using the fMdPEG4C antigen showed low antigen-binding ability to proteins containing a small amount of formylmethionine increased by actinonin treatment, whereas antibodies produced using the mixed antigen fMXC showed significantly higher antigen-binding ability than these antibodies (FIG. 2).

Through this, it was confirmed that the antibodies produced using the mixed antigen fMXC of the present disclosure had superior sensitivity to formylmethionine than the conventional antibodies.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

### Sequence List Free Text

Sequence information for SEQ ID NOs: 1 to 21 having less than 4 defined amino acids as described herein is provided below.

| SEQ ID NO | Sequence | Type of Sequence | Name of Organism |
|---|---|---|---|
| 1 | fMXC | Amino acid | synthetic construct |
| 2 | fMGC | Amino acid | synthetic construct |
| 3 | fMAC | Amino acid | synthetic construct |
| 4 | fMVC | Amino acid | synthetic construct |
| 5 | fMLC | Amino acid | synthetic construct |
| 6 | fMIC | Amino acid | synthetic construct |
| 7 | fMTC | Amino acid | synthetic construct |
| 8 | fMSC | Amino acid | synthetic construct |
| 9 | fMCC | Amino acid | synthetic construct |
| 10 | fMMC | Amino acid | synthetic construct |
| 11 | fMDC | Amino acid | synthetic construct |
| 12 | fMNC | Amino acid | synthetic construct |
| 13 | fMEC | Amino acid | synthetic construct |
| 14 | fMQC | Amino acid | synthetic construct |
| 15 | fMKC | Amino acid | synthetic construct |
| 16 | fMRC | Amino acid | synthetic construct |
| 17 | fMHC | Amino acid | synthetic construct |
| 18 | fMFC | Amino acid | synthetic construct |
| 19 | fMYC | Amino acid | synthetic construct |
| 20 | fMWC | Amino acid | synthetic construct |
| 21 | fMPC | Amino acid | synthetic construct |

*fM: Formylmethionine (fMet, fM)

### 3-3. Antigen-Binding Ability of Antibodies in Purified Proteins

To confirm the antibody sensitivity of the three types of formylmethionine-specific antibodies (anti-fMGSGC serum, anti-fMdPEG4C serum, and anti-fMXC serum) produced in Example 2 in purified proteins, an enzyme-linked immunosorbent assay (ELISA) was performed. Specifically, MD-D2³⁻¹¹-GST (MD: Methionine-Aspartate; D2³⁻¹¹: Amino acids 3-11 of the D2 protein from Chlamydomonas reinhardtii; GST: Glutathione-S-transferase) and fMD-D2³⁻¹¹-GST (fMD: N-formyl-Methionine-Aspartate; D2³⁻¹¹: Amino acids 3-11 of the D2 protein from Chlamydomonas reinhardtii; GST: Glutathione-S-transferase) proteins were prepared at a concentration of 1000 nM in 50 mM carbonate coating buffer (45 mM NaHCO₃+5 mM Na₂CO₃). Additionally, the 1000 nM protein samples were diluted in 50 mM carbonate coating buffer to prepare additional protein samples at concentrations of 100 nM, 10 nM, 1 nM, and 0.1 nM. A total of 100 μl of the various concentrations of MD-D2³⁻¹¹-GST protein and fMD-D2³⁻¹¹-GST protein samples were transferred into each well of a 96-well plate (white, non-transparent) and coated overnight at 4° C. Afterward, each well was washed three times with PBS-T and blocked with 3% BSA (bovine serum albumin) in PBS-T at 37° C. for 1 hour. The wells were then washed three more times with PBS-T, and the formylmethionine-specific antibody (unpurified anti-fMXC serum) produced in Example 2, as well as two control antibodies (anti-fMGSGC serum and anti-fMdPEG4C serum), were each diluted in 3% BSA in PBS-T at a 1:1000 ratio and reacted at 37° C. for 2 hours. Each well was washed again three times with PBS-T, and then a secondary antibody (goat anti-rabbit IgG HRP conjugate, diluted 1:10000 in 3% BSA in PBS-T) was reacted at 37° C. for 1 hour. After washing each well five times with PBS-T, detection was performed using the SuperSignal ELISA Femto Substrate reagent and Promega GloMax equipment.

As a result, the formylmethionine antibody produced using the mixed antigen fMXC was found to specifically bind to the protein containing formylmethionine (FIG. 3). While the antibodies produced using the conventional fMGSGC antigen and the antibodies produced using the fMdPEG4C antigen exhibited low antibody sensitivity, the antibody produced using the mixed antigen fMXC showed significantly higher antibody sensitivity compared to these (FIG. 3).

What is claimed is:

1. An antigen peptide for producing a formylmethionine (fMet, fM)-specific antibody comprising an amino acid sequence of Formula 1 below:
fM-X-C (Formula 1, SEQ ID NO: 1)
wherein,
fM is formylmethionine; and
X is G (Gly), A (Ala), V (Val), L (Leu), I (Ile), T (Thr), S (Ser), C (Cys), M (Met), D (Asp), N (Asn), E (Glu), Q (Gln), K (Lys), R (Arg), H (His), F (Phe), Y (Tyr), W (Trp), or P (Pro).

2. The antigen peptide for producing the formylmethionine-specific antibody of claim 1, wherein X is at least one selected from the group consisting of G (Gly), A (Ala), V (Val), L (Leu), I (Ile), T (Thr), S (Ser), C (Cys), M (Met), D (Asp), N (Asn), E (Glu), Q (Gln), K (Lys), R (Arg), H (His), F (Phe), Y (Tyr), W (Trp), and P (Pro).

3. The antigen peptide for producing the formylmethionine-specific antibody of claim 1, wherein the antigen peptide comprises a peptide including an amino acid sequence of fMGC, a peptide including an amino acid sequence of fMAC, a peptide including an amino acid sequence of fMVC, a peptide including an amino acid sequence of fMLC, a peptide including an amino acid sequence of fMIC, a peptide including an amino acid sequence of fMTC, a peptide including an amino acid sequence of fMSC, a peptide including an amino acid sequence of fMCC, a peptide including an amino acid sequence of fMMC, a peptide including an amino acid sequence of fMDC, a peptide including an amino acid sequence of fMNC, a peptide including an amino acid sequence of fMEC, a peptide including an amino acid sequence of fMQC, a peptide including an amino acid sequence of fMKC, a peptide including an amino acid sequence of fMRC, a peptide including an amino acid sequence of fMHC, a peptide including an amino acid sequence of fMFC, a peptide including an amino acid sequence of fMYC, a peptide including an amino acid sequence of fMWC, and a peptide including an amino acid sequence of fMPC.

4. The antigen peptide for producing the formylmethionine-specific antibody of claim 1, further comprising:
a carrier protein.

5. The antigen peptide for producing the formylmethionine-specific antibody of claim 4, wherein the carrier protein is linked via a linker.

6. The antigen peptide for producing the formylmethionine-specific antibody of claim 5, wherein the linker is sulfosuccinimidyl 4-(N-maleimidomethyl)cyclohexane-1-carboxylate (sulfo-SMCC).

7. A composition for producing a formylmethionine-specific antibody comprising the antigen peptide of claim 1.

8. The composition for producing the formylmethionine-specific antibody of claim 7, wherein the composition comprises a peptide including an amino acid sequence of fMGC, a peptide including an amino acid sequence of fMAC, a peptide including an amino acid sequence of fMVC, a peptide including an amino acid sequence of fMLC, a peptide including an amino acid sequence of fMIC, a peptide including an amino acid sequence of fMTC, a peptide including an amino acid sequence of fMSC, a peptide including an amino acid sequence of fMCC, a peptide including an amino acid sequence of fMMC, a peptide including an amino acid sequence of fMDC, a peptide including an amino acid sequence of fMNC, a peptide including an amino acid sequence of fMEC, a peptide including an amino acid sequence of fMQC, a peptide including an amino acid sequence of fMKC, a peptide including an amino acid sequence of fMRC, a peptide including an amino acid sequence of fMHC, a peptide including an amino acid sequence of fMFC, a peptide including an amino acid sequence of fMYC, a peptide including an amino acid sequence of fMWC, and a peptide including an amino acid sequence of fMPC.

9. A method for producing a formylmethionine-specific antibody comprising:
a) inducing an immune response by inoculating the antigen peptide of claim 1 multiple times to a host other than a human; and
b) obtaining serum from the blood of the host.

10. The method for producing the formylmethionine-specific antibody of claim 9, further comprising:
purifying the formylmethionine antibody from the serum.

11. A method for producing a formylmethionine-specific antibody comprising:
a) inducing an immune response by inoculating the composition of claim 7 multiple times to a host other than a human; and
b) obtaining serum from the blood of the host.

12. The method for producing the formylmethionine-specific antibody of claim 11, further comprising:
purifying the formylmethionine antibody from the serum.
